(19) United States
(12) Patent Application Publication
(10) Pub. No.: US 2008/0206367 A1
(43) Pub. Date: Aug. 28, 2008

(54) Morinda Citrifolia-based Formulations and Methods for Weight Management

(76) Inventors: Sid Liu, Shanghai (CN); Chen XIng Su, West Jordan, UT (US); Lois Lo, Taipei (TW); Claude Jarake Jensen, Cedar Hills, UT (US); Stephen P. Story, Alpine, UT (US)

Correspondence Address: KIRTON AND MCCONKIE, 60 EAST SOUTH TEMPLE,, SUITE 1800, SALT LAKE CITY, UT, 84111, US

(21) Appl. No.: 11/925,035

(22) Filed: Oct. 26, 2007

Related U.S. Application Data
(62) Division of application No. 11/216,418, filed on Aug. 30, 2005, now abandoned.
(60) Provisional application No. 60/606,531, filed on Sep. 1, 2004.

(51) Int. Cl. A61K 36/45; A61P 3/04

(52) U.S. Cl. 424/732

(57) ABSTRACT

The present invention relates to formulations and methods for weight management utilizing processed Morinda citrifolia products or extracts. Specifically, the present invention relates to formulations, which may be used for weight loss, regulating gastric motility and regulating plasma levels of cholecystokinin.

### RELATED APPLICATIONS

This application claims priority to U.S. Provisional Patent Application Ser. No. 60/636,531, filed Sep. 1, 2004.

### BACKGROUND OF THE INVENTION

1. Field of the Invention

The present invention relates to formulations and methods for weight management, which utilize processed Morinda citrifolia products. Specifically, the present invention relates to formulations and methods for weight management including biochemical mechanisms comprised of regulation of hormones comprised of cholecystokinin (“CCK”).

2. Background and Related Art

It has been estimated that 30% to 35% of Americans are overweight or obese. Obesity has been defined as a weight more than 20% above what is considered normal according to standard age, height, and weight tables, or by a complex formula known as the body mass index.

Research related to weight control has yielded a complicated picture of the underlying causes of being overweight or obese. The simple cause is ingestion of more calories than are required for energy, the excess being stored in the body as fat. Inactivity and insufficient exercise can be contributing factors; the less active the person, the fewer calories are needed to maintain normal body weight. Overeating may result from unhealthful patterns of eating established by the family and cultural environment, perhaps exacerbated by psychological distress, an emotional dependence on food, or the omnipresence of high-calorie foods.

In some cases, being overweight or obese can come from an eating disorder. It has been shown, for example, that binging for some people releases natural opiates in the brain, providing a sense of well-being and physical pleasure. Other studies have found a strong relationship between obesity or being overweight in women and childhood sexual abuse.

Some weight-loss experts see obesity as based upon genetics and physiology rather than as a behavioral or psychological problem. For example, rat studies have shown that fat cells secrete a hormone that helps the rat's brain assess the amount of body fat present. The brain tries to keep the amount of that hormone (which also appears to act on the brain area that regulates appetite and metabolic rate) at a set level, resulting in the so-called set point—a weight that the body comes back to, even after resolute dieting. The gene that encodes this hormone, called the obese or ob gene, has been isolated in both rats and humans. In addition, a gene that influences obesity and the onset of diabetes has been identified. It has been estimated that from 8 to 30 different genes may influence obesity.

Obesity, and more generally being overweight, is a major public health concern because it predisposes the individual to many disorders, such as noninsulin-dependent diabetes, hypertension, stroke, and coronary artery disease, and has been associated with an increased incidence of certain cancers, notably cancers of the colon, rectum, prostate, breast, uterus, and cervix. In contemporary American society, being overweight also carries with it a sometimes devastating social stigma. Overweight people are often ostracized, and discrimination against them, especially in hiring and promotion, is common.

Radical treatments for weight loss have included wiring shut the jaw, stapling the stomach, and intestinal bypass operations circumventing a large area of the small intestine, limiting the area where food is absorbed. The “diet pills” of the 1960s, essentially amphetamines such as Dexedrine, are now seldom prescribed for weight loss. Fenfluramine and dexfenfluramine, drugs formerly used to achieve short-term weight loss, were withdrawn from the market following concerns that they could cause heart valve damage. Drugs available in the late 1990s included sibutramine (Meridia), which is an appetite suppressant, and orlistat (Xenical), which acts to block absorption of dietary fat in the intestine.

Although the study of obesity is yielding many possibilities for treatment, the main focus remains diet (especially a diet limiting fat calories) and exercise, often coupled with emotional and behavioral support. The long-term weight-loss success of most attempts at dieting, however, is notoriously low. Groups such as Overeaters Anonymous, modeled after Alcoholics Anonymous, give support to people with weight problems and eating disorders.

### SUMMARY OF THE INVENTION

The present invention relates to weight management utilizing processed Morinda citrifolia products. In some embodiments Morinda citrifolia is administered to a subject to inhibit gastric emptying. In some embodiments, Morinda citrifolia is administered to a subject to increase plasma CCK concentration.

These and other features and advantages of the present invention will be set forth or will become more fully apparent in the description that follows and in the appended claims. The features and advantages may be realized and obtained by means of the instruments and combinations particularly pointed out in the appended claims. Furthermore, the features and advantages of the invention may be learned by the practice of the invention or will be obvious from the description, as set forth hereinafter.

### BRIEF DESCRIPTION OF THE DRAWINGS

In order that the manner in which the above recited and other features and advantages of the present invention are obtained, a more particular description of the invention will be rendered by reference to specific embodiments thereof, which are illustrated in the appended drawings. Understanding that the drawings depict only typical embodiments of the present invention and are not, therefore, to be considered as limiting the scope of the invention, the present invention will be described and explained with additional specificity and detail through the use of the accompanying drawings in which:

FIG. 1 depicts graphically water intake, food intake and body weight over a seven day period of time;

FIG. 2 depicts graphically measurements for feces weight, feces and urine volume over a seven day period of time;

FIG. 3 depicts graphically gastric emptying versus the amount of Morinda Citrifolia ingested, and intestinal transit versus the amount of Morinda Citrifolia ingested;

FIG. 4 depicts graphically gastric emptying versus the amount of Morinda Citrifolia ingested, and intestinal transit versus the amount of Morinda Citrifolia ingested;

FIG. 5 depicts graphically gastric emptying versus amount of Morinda Citrifolia and lorglumide consumed, and intestinal transit versus amount of Morinda Citrifolia and lorglumide consumed;

FIG. 6 depicts graphically plasma PNF levels versus amount of Morinda Citrifolia ingested per day; and

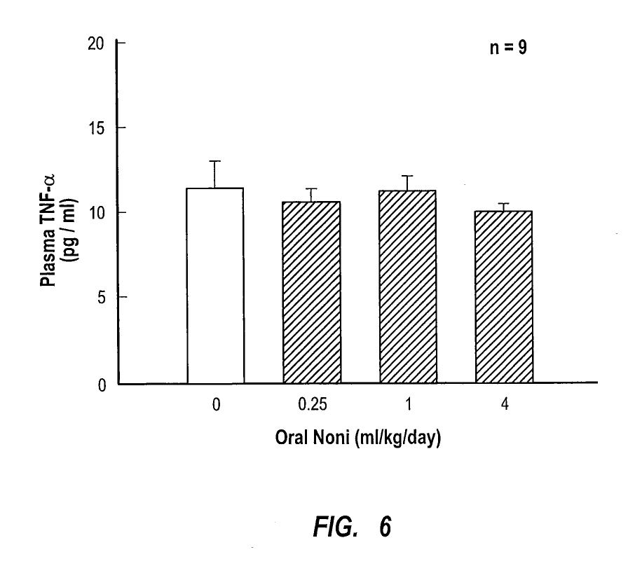

FIG. 7 illustrates plasma CCK (pg/ml) levels versus the amount of Morinda Citrifolia ingested each day.

### DETAILED DESCRIPTION OF THE INVENTION

The present invention relates to nutraceutical formulations and methods for weight management, which utilize processed Morinda citrifolia products.

The following disclosure of the present invention is grouped into subheadings, namely “General Discussion of Morinda citrifolia and the Methods Used to Produce Processed Morinda citrifolia Products” and “Formulations and Methods of Administration Morinda citrifolia for Weight Management.” The utilization of the subheadings is for convenience of the reader only and is not to be construed as limiting in any sense.

1. General Discussion of Morinda citrifolia and the Methods Used to Produce Processed Morinda citrifolia Products

The Indian Mulberry or Noni plant, known scientifically as Morinda Citrifolia L. (Morinda citrifolia), is a shrub or small tree up to 10 m in height. The leaves are oppositely arranged with an elliptic to ovate form. The small white flowers are contained in a fleshy, globose, head-like cluster. The fruits are large, fleshy, and ovoid. At maturity, they are creamy-white and edible, but have an unpleasant taste and odor. The plant is native to Southeast Asia and has spread in early times to a vast area from India to eastern Polynesia. It grows randomly in the wild, and it has been cultivated in plantations and small individual growing plots. The Morinda citrifolia flowers are small, white, three to five lobed, tubular, fragrant, and about 1.25 cm long. The flowers develop into compound fruits composed of many small drupes fused into an ovoid, ellipsoid or roundish, lumpy body, with waxy, white, or greenish-white or yellowish, semi-translucent skin. The fruit contains “eyes” on its surface, similar to a potato. The fruit is juicy, bitter, dull-yellow or yellowish-white, and contains numerous red-brown, hard, oblong-triangular, winged 2-celled stones, each containing four seeds.

When fully ripe, the fruit has a pronounced odor like rancid cheese. Although the fruit has been eaten by several nationalities as food, the most common use of the Morinda citrifolia plant was as a red and yellow dye source. Recently, there has been an interest in the nutritional and health benefits of the Morinda citrifolia plant, further discussed below.

Because the Morinda citrifolia fruit is for all practical purposes inedible, the fruit must be processed in order to make it palatable for human consumption and included in the nutraceutical used to regulate mammalian body weight. Processed Morinda citrifolia fruit juice can be prepared by separating seeds and peels from the juice and pulp of a ripened Morinda citrifolia fruit; filtering the pulp from the juice; and packaging the juice. Alternatively, rather than packaging the juice, the juice can be immediately included as an ingredient in another food product, frozen or pasteurized. In some embodiments, the juice and pulp can be pureed into a homogenous blend to be mixed with other ingredients. Other process include freeze drying the fruit and juice. The fruit and juice can be reconstituted during production of the final juice product. Still other processes include air drying the fruit and juices, prior to being masticated.

The present invention also contemplates the use of fruit juice and/or puree fruit juice extracted from the Morinda Citrifolia plant. In a currently preferred process of producing Morinda citrifolia fruit juice, the fruit is either hand picked or picked by mechanical equipment. The fruit can be harvested when it is at least one inch (2-3 cm) and up to 12 inches (24-36 cm) in diameter. The fruit preferably has a color ranging from a dark green through a yellow-green up to a white color, and gradations of color in between. The fruit is thoroughly cleaned after harvesting and before any processing occurs.

The fruit is allowed to ripen or age from 0 to 14 days, with most fruit being held from 2 to 3 days. The fruit is ripened or aged by being placed on equipment so it does not contact the ground. It is preferably covered with a cloth or netting material during aging, but can be aged without being covered. When ready for further processing the fruit is light in color, from a light green, light yellow, white or translucent color. The fruit is inspected for spoilage or for excessively green color and hard firmness. Spoiled and hard green fruit is separated from the acceptable fruit.

The ripened and aged fruit is preferably placed in plastic lined containers for further processing and transport. The containers of aged fruit can be held from 0 to 120 days. Most fruit containers are held for 7 to 14 days before processing. The containers can optionally be stored under refrigerated conditions or ambient/room temperature conditions prior to further processing. The fruit is unpacked from the storage containers and is processed through a manual or mechanical separator. The seeds and peel are separated from the juice and pulp.

The juice and pulp can be packaged into containers for storage and transport. Alternatively, the juice and pulp can be immediately processed into a finished juice product. The containers can be stored in refrigerated, frozen, or room temperature conditions.

The Morinda citrifolia juice and pulp are preferably blended in a homogenous blend, after which they may be mixed with other ingredients, such as flavorings, sweeteners, nutritional ingredients, botanicals, and colorings. The finished juice product is preferably heated and pasteurized at a minimum temperature of 181° F. (83° C.) or higher up to 212° F. (100° C.).

Another product manufactured is Morinda citrifolia puree and puree juice, in either concentrate or diluted form. Puree is essentially the pulp separated from the seeds and is different than the fruit juice product described herein.

Each product is filled and sealed into a final container of plastic, glass, or another suitable material that can withstand the processing temperatures. The containers are maintained at the filling temperature or may be cooled rapidly and then placed in a shipping container. The shipping containers are preferably wrapped with a material and in a manner to maintain or control the temperature of the product in the final containers.

The juice and pulp may be further processed by separating the pulp from the juice through filtering equipment. The filtering equipment preferably consists of, but is not limited to, a centrifuge decanter, a screen filter with a size from 0.01 micron up to 2000 microns, more preferably less than 500 microns, a filter press, reverse osmosis filtration, and any other standard commercial filtration devices. The operating filter pressure preferably ranges from 0.1 psig up to about 1000 psig. The flow rate preferably ranges from 0.1 g.p.m. up to 1000 g.p.m., and more preferably between 5 and 50 g.p.m. The wet pulp is washed and filtered at least once and up to 10 times to remove any juice from the pulp. The wet pulp typically has a fiber content of 10 to 40 percent by weight. The wet pulp is preferably pasteurized at a temperature of 181° F. (83° C.) minimum and then packed in drums for further processing or made into a high fiber product.

The processed Morinda citrifolia product may also exist as a dietary fiber. Still further, the processed Morinda citrifolia product may also exist in oil form. The Morinda citrifolia oil typically includes a mixture of several different fatty acids as triglycerides, such as palmitic, stearic, oleic, and linoleic fatty acids, and other fatty acids present in lesser quantities. In addition, the oil preferably includes an antioxidant to inhibit spoilage of the oil. Conventional food grade antioxidants are preferably used.

The Morinda citrifolia plant is rich in natural ingredients. Those ingredients that have been discovered include: (from the leaves): alanine, anthraquinones, arginine, ascorbic acid, aspartic acid, calcium, beta-carotene, cysteine, cystine, glycine, glutamic acid, glycosides, histidine, iron, leucine, isoleucine, methionine, niacin, phenylalanine, phosphorus, proline, resins, riboflavin, serine, beta-sitosterol, thiamine, threonine, tryptophan, tyrosine, ursolic acid, and valine; (from the flowers): acacetin-7-o-beta-d(+)-glucopyranoside, 5,7-dimethyl-apigenin-4′-o-beta-d(+)-galactopyranoside, and 6,8-dimethoxy-3-methylanthraquinone-1-o-beta-rhamnosyl-glucopyranoside; (from the fruit): acetic acid, asperuloside, butanoic acid, benzoic acid, benzyl alcohol, 1-butanol, caprylic acid, decanoic acid, (E)-6-dodeceno-gamma-lactone, (Z,Z,Z)-8,11,14-eicosatrienoic acid, elaidic acid, ethyl decanoate, ethyl hexanoate, ethyl octanoate, ethyl palmitate, (Z)-6-(ethylthiomethyl) benzene, eugenol, glucose, heptanoic acid, 2-heptanone, hexanal, hexanamide, hexanedioic acid, hexanoic acid (hexoic acid), 1-hexanol, 3-hydroxy-2-butanone, lauric acid, limonene, linoleic acid, 2-methylbutanoic acid, 3-methyl-2-buten-1-ol, 3-methyl-3-buten-1-ol, methyl decanoate, methyl elaidate, methyl hexanoate, methyl 3-methylthio-propanoate, methyl octanoate, methyl oleate, methyl palmitate, 2-methylpropanoic acid, 3-methylthiopropanoic acid, myristic acid, nonanoic acid, octanoic acid (octoic acid), oleic acid, palmitic acid, potassium, scopoletin, undecanoic acid, (Z,Z)-2,5-undecadien-1-ol, and vomifol; (from the roots): anthraquinones, asperuloside (rubichloric acid), damnacanthal, glycosides, morindadiol, morindine, morindone, mucilaginous matter, nor-damnacanthal, rubiadin, rubiadin monomethyl ether, resins, soranjidiol, sterols, and trihydroxymethyl anthraquinone-monomethyl ether; (from the root bark): alizarin, chlororubin, glycosides (pentose, hexose), morindadiol, morindanigrine, morindine, morindone, resinous matter, rubiadin monomethyl ether, and soranjidiol; (from the wood): anthragallol-2,3-dimethylether; (from the tissue culture): damnacanthal, lucidin, lucidin-3-primeveroside, and morindone-6beta-primeveroside; (from the plant): alizarin, alizarin-alpha-methyl ether, anthraquinones, asperuloside, hexanoic acid, morindadiol, morindone, morindogenin, octanoic acid, and ursolic acid. The present invention contemplates utilizing all parts of the M. citrifolia plant alone, in combination with each other or in combination with other ingredients. The above listed portions of the M. citrifolia plant is not an exhaustive list of parts of the plant to be used but are merely exemplary. Thus, while some of the parts of the M. citrifolia plant are not mentioned above (e.g., seed from the fruit, the pericarp of the fruit, the bark or the plant) the present invention contemplates the use of all of the parts of the plant.

The compositions containing Morinda citrifolia may be in a form suitable for oral use, for example, as tablets, or lozenges, aqueous or oily suspensions, dispersible powders or granules, emulsions, syrups or elixirs. Compositions intended for oral use may be prepared according to any method known in the art for the manufacture of Morinda citrifolia compositions and such compositions may contain one or more agents selected from the group consisting of sweetening agents, flavoring agents, coloring agents and preserving agents. Tablets contain Morinda citrifolia in admixture with non-toxic pharmaceutically acceptable excipients, which are suitable for the manufacture of tablets. These excipients may be for example, inert diluents, granulating and disintegrating agents, binding agents, and lubricating agents. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed.

Aqueous suspensions contain the Morinda citrifolia in admixture with excipients suitable for the manufacture of aqueous suspensions. Such excipients are suspending agents, for example, sodium carboxymethyl-cellulose, methylcellulose, hydroxy-propylmethycellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethylene-oxycetanol, or condensation products of ethylene oxide with partial esters derived from fatty acids and a hexitol such as polyoxyethylene sorbitor monooleate, or condensation products of ethylene oxide with partial esters derived from fatty acids and hexitol anhydrides, for example polyethylene sorbitan monooleate.

Favorably, this invention provides a method weight management with a Morinda citrifolia-based nutraceutical formulation without any significant tendency to cause side effects.

### 2. Formulations and Methods of Administration

The present invention provides formulations and methods for weight management. Specifically, the present invention provides systems and methods for administering a treatment formulated with Morinda citrifolia from the Indian Mulberry plant. The Morinda citrifolia is incorporated into various carriers or nutraceutical compositions suitable for in vivo treatment of a patient. For instance, the processed Morinda citrifolia may be ingested, introduced through an intravenous injection or feeding, or otherwise internalized as is appropriate and directed.

In one exemplary embodiment, the nutraceutical composition of the present invention comprises one or more of a processed Morinda citrifolia product present in an amount by weight between about 0.01 and 100 percent by weight, and preferably between 0.01 and 95 percent by weight. Several embodiment of formulations are provided below. However, these are only intended to be exemplary as one ordinarily skilled in the art will recognize other formulations or compositions comprising the processed Morinda citrifolia product.

In some embodiments, the processed Morinda citrifolia product is the active ingredient or contains one or more active ingredients, such as Quercetin and Rutin, and others, for effectuating natural control of the body weight of mammals. One embodiment of the present invention comprises a processed Morinda citrifolia product that promotes natural weight loss. Active ingredients may be extracted out using various alcohol or alcohol-based solutions, such as methanol, ethanol, and ethyl acetate, and other alcohol-based derivatives using any known process in the art. The active ingredients of Quercetin and Rutin are present in amounts by weight ranging from 0.01-10 percent of the total formulation or composition. These amounts may be concentrated as well into a more potent concentration in which they are present in amounts ranging from 10 to 100 percent.

In some embodiments, the processed Morinda citrifolia product may be formulated with various other ingredients to produce various compositions, such as a nutraceutical composition, an internal composition, or others. The ingredients to be utilized in a nutraceutical composition are any that are safe for introduction into the body of a mammal, and particularly a human, and may exist in various forms, such as liquids, tablets, lozenges, aqueous or oily solutions, dispersible powders or granules, emulsions, syrups, elixirs, etc. Moreover, since the nutraceutical composition will most likely be consumed orally, it may contain one or more agents selected from the group consisting of sweetening agents, flavoring agents, coloring agents, preserving agents, and other medicinal agents as directed.

In some embodiments, the ingredients to be utilized in a topical dermal composition are also any that are safe for internalizing into the body of a mammal and may exist in various forms, such as gels, lotions, creams, ointments, etc., each comprising one or more carrier agents. The ingredients for systemically administered formulations may also comprise any known in the art.

In some embodiments, the present invention further features a method of administering a composition to a mammal for the purpose of weight management. The method comprises the steps of (a) formulating a composition comprising a Morinda citrifolia product present in an amount between about 0.01 and 95 percent by weight; (b) administering the composition to a mammal such that the processed Morinda citrifolia product is sufficiently internalized; (c) repeating the above steps as often as necessary to provide an effective amount of the processed Morinda citrifolia product.

In some embodiments, the step of administering the nutraceutical composition into the body comprises ingesting the composition orally through one of several means. Specifically, the nutraceutical composition may be formulated as a liquid, gel, solid, or some other type that would allow the composition to be quickly and/or conveniently digested. Once sufficiently internalized, the administered nutraceutical composition may then begin to act to manage the weight of the subject. The management of weight may include administration of the nutraceutical composition to promote natural weight loss. The management of weight may include administration of the nutraceutical composition to maintain a desired body weight. Generally, it is contemplated that a broad range of objectives regarding the management of weight accomplished by consumption of products disclosed in the present invention may be accomplished by varying the formulation and administration procedures followed. In addition, the step of administering the nutraceutical composition may include injecting the composition into the body using an intravenous pump.

In one exemplary embodiment, the nutraceutical composition is administered by taking between 1 teaspoon and 2 oz., and preferably 2 oz., of the nutraceutical composition every two hours each day, or at least twice a day. The nutraceutical composition is to be taken on an empty stomach, meaning at a period of time at least two hours prior to consumption of any food or drink. Of course, one ordinarily skilled in the art will recognize that the amount of composition and frequency of use may vary from individual to individual.

The following tables illustrate or represent some of the preferred formulations or compositions contemplated by the present invention. As stated, these are only intended as exemplary embodiments and are not to be construed as limiting in any way.

### Formulation One

| Ingredients | Percent by Weight |
|---|---|
| Morinda citrifolia puree juice or fruit juice | 100% |

### Formulation Two

| Ingredients | Percent by Weight |
|---|---|
| Morinda citrifolia fruit juice | 50-99.99% |
| water | 0.1-50% |

### Formulation Three

| Ingredients | Percent by Weight |
|---|---|
| Morinda citrifolia fruit juice | 50-99.99% |
| non-Morinda citrifolia-based fruit juices | 0.1-50% |

### Formulation Four

| Ingredients | Percent by Weight |
|---|---|
| Morinda citrifolia fruit juice | 50-90% |
| water | 0.1-50% |
| non-Morinda citrifolia-based fruit juices | 0.1-30% |

### Formulation Five

| Ingredients | Percent by Weight |
|---|---|
| Morinda citrifolia puree juice | 50-99.9% |
| water | 0.1-50% |

### Formulation Six

| Ingredients | Percent by Weight |
|---|---|
| Morinda citrifolia puree juice | 50-99.9% |
| non-Morinda citrifolia-based fruit juices | 0.1-50% |
| (comprising blueberry and grape juice) | |

### Formulation Seven

| Ingredients | Percent by Weight |
|---|---|
| Morinda citrifolia puree juice | 50-90% |
| water | 0.1-50% |
| non-Morinda citrifolia-based fruit juices | 0.1-30% |

### Formulation Eight

| Ingredients | Percent by Weight |
|---|---|
| Morinda citrifolia dietary fiber | 0.1-50% |
| water | 1-99.9% |
| non-Morinda citrifolia-based fruit juices | 1-99.9% |

### Formulation Nine

| Ingredients | Percent by Weight |
|---|---|
| Morinda citrifolia dietary fiber | 0.1-50% |
| water | 1-99.9% |
| Morinda citrifolia fruit juice or puree juice | 1-99.9% |

### Formulation Ten

| Ingredients | Percent by Weight |
|---|---|
| Morinda citrifolia oil | 0.1-50% |
| carrier medium | 70-99.9% |
| other ingredients | 1-95% |

### Formulation Eleven

| Ingredients | Percent by Weight |
|---|---|
| Morinda citrifolia product | 10-80% |
| carrier medium | 20-90% |

### Formulation Twelve

| Ingredients | Percent by Weight |
|---|---|
| Morinda citrifolia product | 5-80% |
| carrier medium | 20-95% |

### Formulation Thirteen

| Ingredients | Percent by Weight |
|---|---|
| Morinda citrifolia oil or oil extract | 0.1-50% |
| carrier medium | 20-90% |

### Formulation Fourteen

| Ingredients | Percent by Weight |
|---|---|
| Morinda citrifolia puree juice or fruit Juice | 0.1-80% |
| Morinda citrifolia oil | 0.1-50% |
| carrier medium | 20-90% |

### Formulation Fifteen

| Ingredients | Percent by Weight |
|---|---|
| Morinda citrifolia puree juice concentrate | 100% |
| or fruit juice concentrate | |

### Formulation Sixteen

| Ingredients | Percent by Weight |
|---|---|
| Morinda citrifolia fruit juice concentrate | 50-99.99% |
| or puree juice concentrate | |
| water | 0.1-50% |

In some embodiments, a person wanting to manage their weight as described above takes, or is administered, at least one (1) ounce of Formulation One in the morning on an empty stomach, and at least one (1) ounce at night on an empty stomach, just prior to retiring to bed. In one example, which is not meant to be limiting in any way, the beneficial Morinda Citrifolia is processed into TAHITIAN NONI® juice manufactured by Morinda, Incorporated of Orem, Utah.

In some embodiments, the present invention features a method for introducing an internal composition of formulation to a subject for the purpose of weight management. In some embodiments, this method comprises the introduction of an internal composition, by oral consumption or otherwise, to the subject for the purpose of weight loss. Several embodiments of the internal comprising various different ingredients are contemplated for use herein, with each embodiment comprising one or more forms of a processed Morinda citrifolia product as taught and explained herein and a carrier agent or medium.

In some embodiments, the internal composition comprises the ingredients of: a processed Morinda citrifolia product present in an amount by weight between about 10-80 percent; and a carrier medium present in an amount by weight between about 20-90 percent. In this embodiment, the processed Morinda citrifolia product may comprise one or more of processed Morinda citrifolia fruit juice, processed Morinda citrifolia puree juice, processed Morinda citrifolia dietary fiber, and/or processed Morinda citrifolia oil extract.

In other embodiments, the internal composition comprises the ingredients of: processed Morinda citrifolia fruit juice or puree juice present in an amount by weight between about 0.1-80 percent; processed Morinda citrifolia oil present in an amount by weight between about 0.1-20 percent; and a carrier medium present in an amount by weight between about 20-90 percent. Morinda citrifolia puree juice or fruit juice may also be formulated with a Morinda citrifolia dietary fiber product in similar concentrations.

In other embodiments Morinda citrifolia is administered at 0.25 ml/kg, 1 ml/kg or 4 ml/kg, for a series of days to accomplish the desired weight control.

According to the present invention, these particular methods of introducing an internal composition may comprise any method of actually introducing the internal composition to the subject for the purpose of weight management. Although the particular methods are many, the present invention recognizes that the internal composition may be introduced intravenously, transdermally, orally, or systemically. No matter what method is employed, it is important to regulate the amount of active ingredient that the subject is exposed to so that the appropriate weight management objectives are accomplished.

In some embodiments, the carrier medium may comprise any ingredient capable of being introduced into the body of a mammal, and that is also capable of providing the carrying medium to the processed Morinda citrifolia product. Specific carrier mediums formulations are well known in the art and are not described in detail herein. In some embodiments, the purpose of the carrier medium is as stated, to provide a means to embody the processed Morinda citrifolia product within the internal composition that is capable of being introduced into the body of the subject to be treated.

The following examples set forth and present the effects of Morinda citrifolia on the management of weight. These examples are not intended to be limiting in any way, but are merely illustrative of the benefits and advantages of utilizing Morinda citrifolia to regulate body weight.

### EXAMPLE 1

### Gastric Motility and Plasma Levels of Cholecystokinin

The present invention relates to nutraceutical formulations and methods for weight regulation utilizing processed Morinda citrifolia products. One embodiment of the present invention comprises the oral administration of Morinda citrifolia products, which increases plasma and cellular levels of CCK to regulate gastric motility.

The effects of juice from Morinda Citrifolia (noni) on gastric emptying, gastrointestinal transit, and plasma level of cholecystokinin (CCK) in rats were studied. Male rats were given noni by gavage at levels of 0.25, 1, or 4 ml/kg once per day for one or 7 days. The rats in the control group were given water, while the rats in the experimental group were fasted overnight before measurement of gastrointestinal motility. Gastrointestinal motility was assessed in rats 15 min after intragastric instillation of a test meal containing charcoal (10%) and Na₂⁵¹CrO₄ (0.5 μCi/ml). Gastric emptying was determined by measuring the amount of radiolabeled chromium contained in the small intestine as a percentage of the initial amount received. Then, gastrointestinal transit was evaluated by calculating the geometric center of distribution of the radiolabeled marker. Finally, blood samples were collected for measurement of CCK by radioimmunoassay. The administration of noni at 0.25 ml/kg, but not at 1 ml/kg and 4 ml/kg, for 1 day significantly inhibited gastric emptying. In contrast, gastric emptying was significantly inhibited by oral noni (0.25, 1, or 4 ml/kg) for 7 days. Intraperitoneal injection of lorglumide (5 or 10 mg/kg), a selective CCK₁ receptor antagonist, effectively attenuated the noni-induced inhibition of gastric emptying. The intestinal transit and body weight, food intake, water intake, urine volume as well as feces weight were not altered by the administration of noni either acutely or chronically, but the administration of oral noni (1 ml/kg) for 7 days increased the level of plasma CCK in male rats. These results suggest that oral noni inhibits gastric emptying in male rats via a mechanism involving stimulation of CCK secretion and CCK₁ receptor activation.

Several experiments were performed that demonstrate the efficacy of the present invention. The following are exemplary demonstrations of the efficacy of the present invention, and are not considered limiting. It would be appreciated by one skilled in the art that the following protocols could be varied to produce various forms of weigh management. Studies were performed to investigate the effects of oral noni on gastric emptying, intestinal transit, and plasma CCK levels, and the involvement of CCK receptors in the action of oral noni on gastrointestinal (GI) motility in male rats by using antagonists of CCK₁, lorglumide.

### Experiment 1. Chronic Effects of Noni Administration on GI Motility

Male rats were randomly divided into four groups and fed with noni (0.25 ml/kg, 1 ml/kg, or 4 ml/kg) via gavage administration once daily for seven days. Noni was provided by Morinda International Inc. The control rats were fed with tap water. All rats were housed in metabolic cages. The body weight, food intake, water intake, urine volume, and feces weight of each rat were recorded daily. Rats were fasted (with access to water) for 24 h before gastric intubation of a non-nutrient liquid meal. Fifteen minutes after the administration of the liquid meal, the rats were decapitated, and GI transit was measured. Blood samples were collected for CCK radioimmunoassay (RIA).

### Experiment 2. Effects of Lorglumide on Noni-Mediated Inhibition of Gastric Emptying

Male rats were divided into four groups and fasted for 24 h before use. Fifteen min before gastric intubation of a non-nutrient liquid meal, the animals were injected i.p. with the following compounds in 1 ml/kg: group 1 received dimethyl sulfoxide (DMSO), while groups 3 and 4 received DMSO containing lorglumide (a CCK₁ receptor antagonist) at doses of 5 and 10 mg/kg, respectively. Groups 2-4 received 1 ml/kg of noni orally once daily for 7 days.

### Experiment 3. Acute Effects of Noni Administration on GI Motility

The procedure was identical to that in experiment 1, except that the oral treatment of noni was performed for one day only.

### Measurement of Gastric Emptying and GI Transit

Gastric emptying and intestinal transit were measured. Rats were intubated via a catheter with physiological saline (3 ml/kg) containing Na₂⁵¹CrO₄ (0.5 μCi/ml) and 10% charcoal. The test meal was continuously stirred before intubation. Air (0.5 ml) was injected to flush the residual charcoal suspension in the catheter into rat stomach. Fifteen minutes later, the rats were decapitated and the stomach with the attached small intestine was immediately exposed by laparotomy. After ligation of the esophagogastric, gastroduodenal, and ileocaecal junctions, the whole stomach with the attached small intestine was carefully removed and placed on a wooden board to observe the leading edge of the charcoal in the intestine. The small intestine was then divided into ten equal segments, and the radioactivity in the stomach and each segment of small intestine was measured in an automatic gamma counter. Gastric emptying was measured by determining the amount of labeled chromium contained in the small intestine fifteen minutes after intubation, expressed as a percentage of the amount given. Intestinal transit was assessed by calculating the geometric center of distribution of the radioactivity within the 10 segments by summation of the radioactivity in each segment multiplied by the segment number.

### Processing of Plasma

After decapitation, rat blood samples were collected and mixed with EDTA (1 mg/ml of blood) and aprotinin (500 KIU/ml of blood). Plasma was immediately prepared by centrifugation at 1000× g for 30 min at 4° and used for measurement of plasma CCK concentrations. The plasma samples were acidified with an equal volume of 1% trifluoroacetic acid (TFA), and then centrifuged at 2600× g for 20 min at 4°. The SEP-PAK C₁₈ cartridge was equilibrated with 60% acetonitrile in 1% TFA (1 ml), followed by 1% TFA (3 ml, three times). Then the supernatant from the treated plasma sample was applied. After slow washing with 1% TFA (3 ml, twice), the peptide (bound material) was slowly eluted with 3 ml of 60% acetonitrile in 1% TFA. The eluant was collected, lyophilized in a Speed Vac concentrator, and then stored at −80° C. and reconstituted with the appropriate assay buffer before RIA.

### CCK Radioimmunoassay

The CCK concentration in extracted sample was measured by RIA using a rabbit anti-CCK antiserum, and ³H-CCK. In this RIA system, a known amount of unlabeled CCK in a total volume of 0.3 ml of 0.1% gelatin-PBS was incubated at 4° for 24 h with 100 μl of anti-CCK antiserum, diluted 1:2,000 in normal rabbit serum, and 100 μl of [³H]CCK (˜8,000 cpm). Triplicate standard curves with 6 points ranging from 1 to 1,000 pg of unlabeled CCK were included in each assay. Two hundred μl of anti-rabbit gamma-globulin (ARGG) was then added and the incubation continued at 4° for 24 hours. The assay tubes were then centrifuged at 1,000× g for 20 minutes. The pellet was dissolved in 400 μl of 1 N NaOH. Then 80 μl of 5 N HCl was added, and the sample was mixed with 3 ml of liquid scintillation fluid. The radioactivity was counted in an automatic counter. The sensitivity of the CCK RIA was 8 pg of CCK per assay tube. The intra-assay and inter-assay coefficient of variation were 3% and 5%, respectively.

### Statistical Analysis

The data were expressed as the mean value±S.E.M. The treatment means were tested for homogeneity using one-way analysis of variance, and the significance of any difference between means tested. A difference between two means was considered to be statistically significant when P was less than 0.05.

### Effects of Noni Administration on Metabolism in Rats

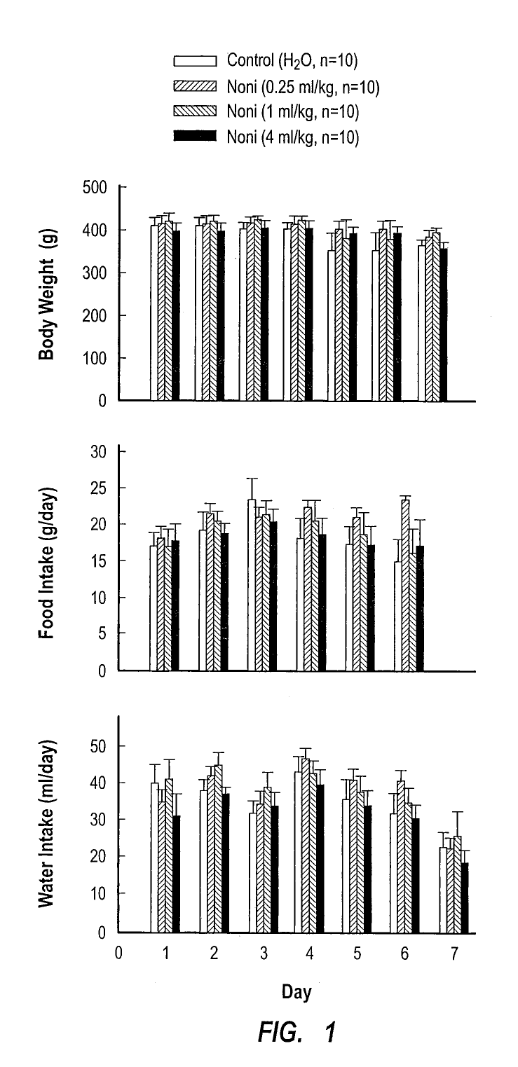

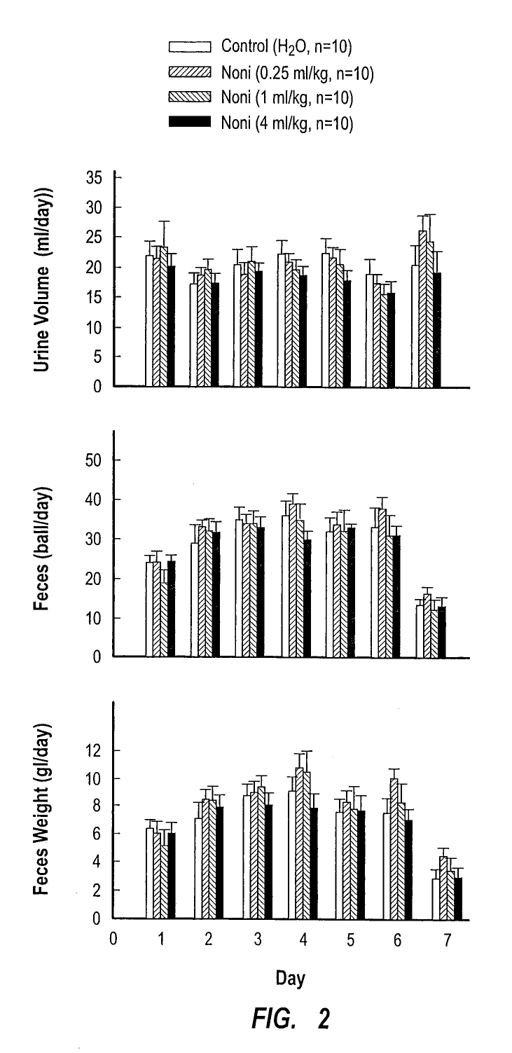

The administration of noni (0.25, 1, and 4 ml/kg) via gavage for one day or 7 days did not alter the body weight, food intake, water intake, urine volume and feces weigh. This data is graphically presented in FIG. 1 and FIG. 2. The water intake and feces weight were reduced following the one-day fast. This data is also graphically presented in FIG. 1 and FIG. 2.

### Acute Effects of Oral Noni on Gastric Emptying and Intestinal Transit

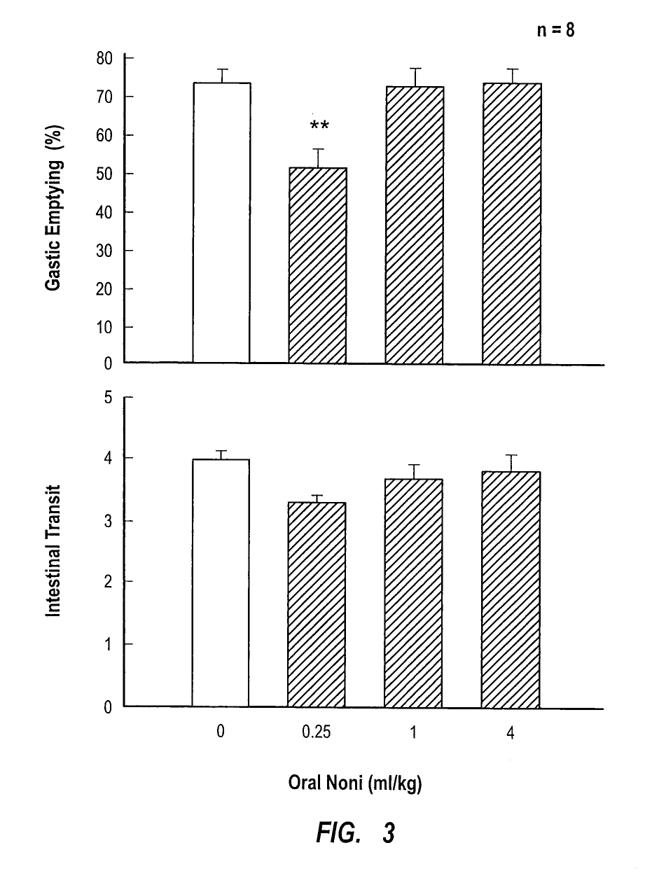

Gastric emptying, but not intestinal transit, in male rats decreased (P<0.01) following oral ingestion of 0.25 ml/kg noni for one day. This data is graphically represented in FIG. 3. Neither gastric emptying nor intestinal transit was altered by oral ingestion of 1 or 4 ml/kg noni for one day.

### Chronic Effects of Oral Noni on Gastric Emptying and Intestinal Transit

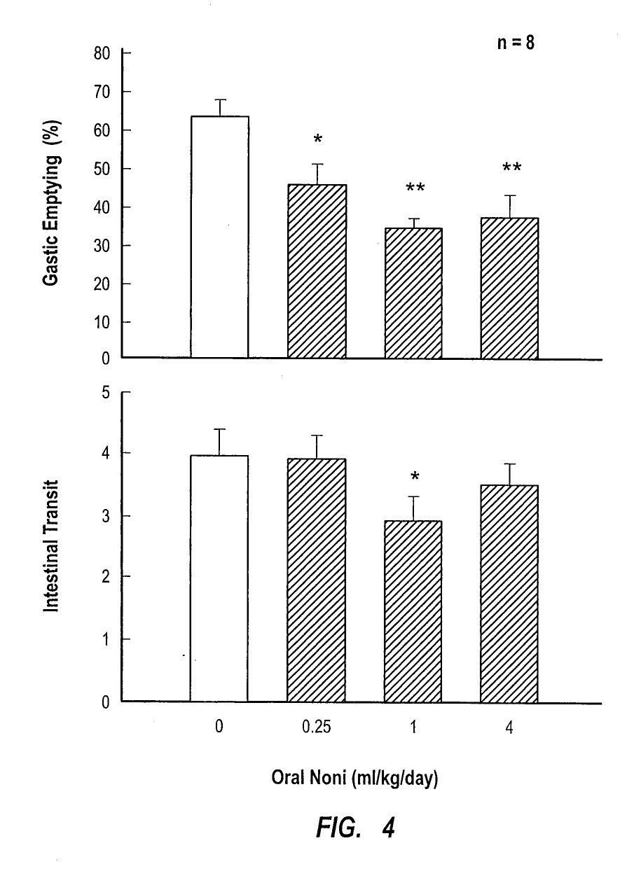

Gastric emptying was reduced by 27% (P<0.05) in male rats following the oral administration of 0.25 ml/kg noni, and by 42-44% (P<0.01) following the administration of 1 or 4 ml/kg noni. (FIG. 4a). Intestinal transit was reduced by oral administration of noni of 1 ml/kg, but not altered by that of 0.25 or 4 ml/kg noni. (FIG. 4b)

### Effects of Lorglumide on Noni-Induced Inhibition of Gastric Emptying

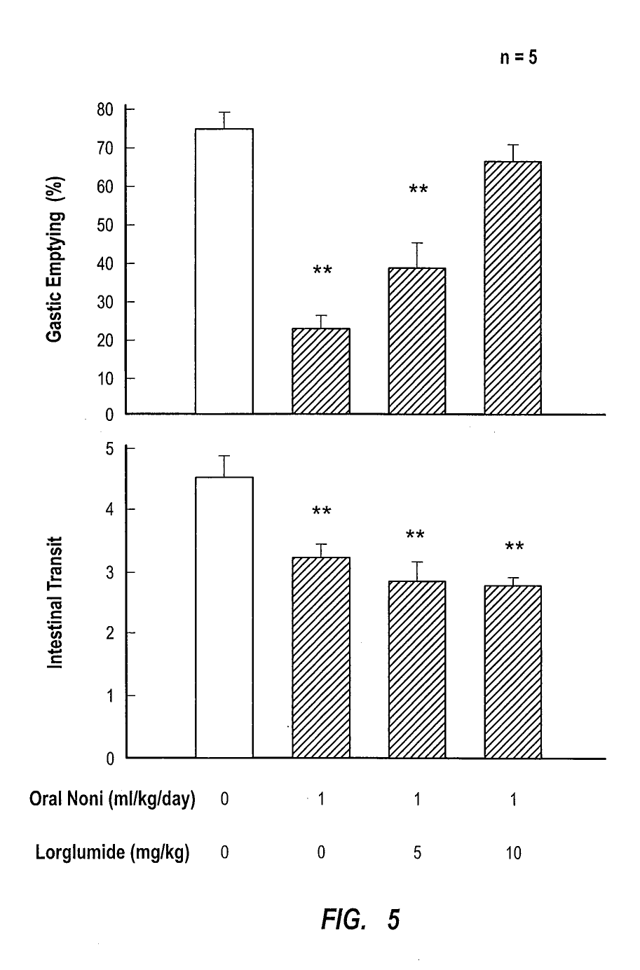

Treatment of lorglumide (5 or 10 ml/kg) significantly prevented (P<0.01) the noni-induced inhibition of gastric emptying (FIG. 5a), and yet the inhibition of intestinal transit caused by noni-induced was not altered by the treatment of lorglumide (FIG. 5b).

### Chronic Effects of Oral Noni on the Level of Plasma CCK

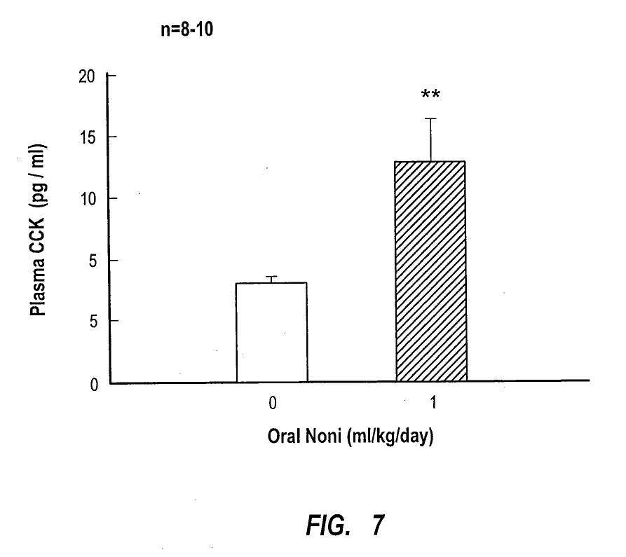

The oral administration of 1 ml/kg noni for 7 days significantly reduced the gastric emptying (FIG. 4), but increased the level of plasma CCK (FIG. 7).

These results demonstrate that the administration of noni inhibited gastric emptying, but increased the plasma CCK concentration in male rats, and the selective CCK₁ receptor antagonist, lorglumide, blocked the noni-induced inhibition of gastric emptying.

Noni has been reported to have a broad range of therapeutic effects. The research conducted regarding the present invention indicates that oral noni administered in a range of 0.25-4 ml/kg decreased gastric emptying, but did not alter intestinal transit in male rats. Since the food intake, water intake, urine volume, and feces weight were not altered by the administration of noni, the change of gastric emptying caused by oral noni was independent of metabolic processes in rats.

It is known that CCK slows gastric emptying in both animals and humans. CCK suppresses food intake by inhibiting gastric emptying. The research that accompanies the present invention indicates that administration of oral noni to male rats resulted in an increase in the plasma CCK level and a marked decrease in gastric emptying. The marked levels of gastric emptying might be related to hypersecretion of CCK. This position is bolstered by the data produced in the CCK antagonist trial. There is now a lot of evidence showing that selective CCK₁ receptor antagonists are able to counterbalance the effects of both exogenous and endogenous CCK. CCK delays gastric emptying of liquids by stimulation of CCK receptors. It is also suggested that CCK inhibits gastric emptying in rats by causing contraction of the pyloric sphincter, which is prevented by CCK₁ receptor antagonists. However, CCK₁ and CCK₂ receptor mRNAs have been detected in the rat stomach and the role of CCK₂ mediating gastric motility has not been established. The present invention is supported by data that shows that lorglumide blocked the noni-induced inhibition of gastric emptying. Apparently, both the actions of CCK and CCK₁ receptor are involved in the regulation of noni on gastric emptying.

In summary, the present invention is supported by research that suggest that oral administration of noni inhibits gastric emptying, which occurred concomittently with an increase of plasma CCK concentration. The results also suggest that CCK₁ receptors are involved in the noni-induced inhibition of gastric emptying. These observations are consistent with the concept that noni, in association with CCK, plays important roles in the regulation of gastric motility.

### EXAMPLE 2

### Regulation of Mammalian Body Weight

The present invention contemplates the use of nutraceutical formulations and methods for regulating mammalian body weight. The present invention contemplates the fact that some individuals will be interested in losing large amounts of weight while others will merely be interested in maintaining their body weight. The present invention contemplates a range of nutraceutical formulations and methods that may accommodate the varying weight regulation interests of specific individuals. The present invention contemplates utilizing variation in ingredients and dosage regimes to accomplish significant or minimal weight loss depending on the needs of the individual.

In an exemplary embodiment of the present invention, individuals could actualize weight loss from 0% of their body weight to nearly 50% of their body weight. This embodiment is supported by research conducted recently. Research performed supports the proposition that certain processed Morinda citrifolia products have a significant impact on weight loss.

In in-vivo assays performed on rats, rats were divided into 4 groups. One group was given water as control. One group was given a dose of 0.25 ml/kg of processed Morinda citrifolia daily—equivalent to 0.5 ounce for a 60 kg human. One group was given a dose of 1 ml/kg of Morinda citrifolia daily—equivalent to 2 ounces for a 60 kg human. The last group was given a dose of 4 ml/kg daily of Morinda citrifolia—equivalent to 8 ounces for a 60 kg human. After 7 days, all of the rats which were administered Morinda citrifolia lost weight compared to the control group. The average weight loss for each group was: 27% for 0.25 ml/kg group, 38% for 1 ml/kg group, and 41% for 4 ml/kg group. The mechanism of such weight loss was correlated with stimulating the secretion of a hormone, cholecystokinin or CCK, in the rat's body. Research performed indicated increased levels of CCK in the rats which had been administered a dose of Morinda citrifolia compared with the control group. These research results indicate that processed Morinda citrifolia has the beneficial effect of regulating weight loss.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes that come within the meaning and range of equivalency of the claims are to be embraced within their scope.

What is claimed is:

1-10. (canceled)

11. A method for regulating mammalian body weight, said method comprising
introducing a processed a composition to a mammal wherein the composition comprises:
Morinda citrifolia juice between about 50 and 99.8% by weight;
grape juice between about 0.1 and 49.9% by weight;
blueberry juice between about 0.1 and 49.9% by weight; and
quercetin present between about 0.01 and 10% by weight; and
rutin present between about 0.01 and 10% by weight; and
repeating the step of introducing the composition to a mammal once a day for a period of seven day.

12. The method of claim 11, further comprising introducing said composition to said mammal for an additional period of time wherein the number of total administrations is selected from a list comprising: 8, 9, 10, 11, 12, 13, 14, 15, 16, 17, 18, 19, and 20.

13. The method of claim 11, wherein the process is repeated daily for more than one month.

14. The method of claim 11, wherein the process is repeated daily for more than one year.

15. The method of claim 11, further comprising introducing said composition to a mammal twice a day for the designated period of time.

16. The method of claim 11, wherein about 1 ml of composition per kilogram of mammalian body weight is introduced to said animal each day.

17. The method of claim 11, wherein about 4.5 ml of composition per kilogram of mammalian body weight is introduced to said mammal each day.

18. The method of claim 11, wherein about 0.25 ml of composition per kilogram of mammalian body weight is introduced to said mammal each day.

19-20. (canceled)

21. The method of claim 11, further comprising combining, prior to administrations, the composition with an item selected from a group consisting of:
Morinda citrifolia oil extract;
Morinda citrifolia dietary fiber;
Morinda citrifolia puree juice;
Morinda citrifolia puree;
Morinda citrifolia fruit juice concentrate; and
Morinda citrifolia puree juice concentrate.
